(19) United States
(12) Patent Application Publication
(10) Pub. No.: US 2011/0092803 A1
(43) Pub. Date: Apr. 21, 2011

(54) NON-INVASIVE DENTAL BASED FIDUCIAL ARRAY

(76) Inventors: Brian Hynes, Montreal (CA); Roch M. Comeau, Montreal (CA); Stephen Frey, Montreal (CA)

(21) Appl. No.: 12/905,882

(22) Filed: Oct. 15, 2010

Related U.S. Application Data
(60) Provisional application No. 61/251,967, filed on Oct. 15, 2009.

(51) Int. Cl. A61B 5/055; A61B 5/05

(52) U.S. Cl. 600/414; 600/426

(57) ABSTRACT

The invention relates generally to apparatus and devices for neuronavigation. A non-invasive dental based fiducial array is disclosed. The fiducial array has a base support member, one or more removable and repositionable marker bases supported by the base support member and a stopper spaced from and supported by the base support member that engages a part of the head of a patient or an animal and cooperates with the base support member to immobilize the fiducial array relative to a location of the head, such as maxillary teeth. An anchoring site is formed on the base support member to accept a custom made dental impression to precisely position the base support member relative to the location of the head.

### CROSS-REFERENCE TO RELATED APPLICATIONS

This application claims priority from U.S. Provisional Application No. 61/251,967 filed on Oct. 15, 2009, hereby incorporated by reference.

### FIELD OF INVENTION

The invention relates generally to apparatus and devices for neuronavigation. More particularly, a non-invasive dental based fiducial array is disclosed.

### BACKGROUND OF INVENTION

It is known to use fiducial arrays to provide fiducial points for neuronavigation. Markers attached to a fiducial array provide the required reference points in an image scan. While fiducial arrays may be attached to a head by employing anchoring holes drilled in the skull, non-invasive fiducial arrays are often preferred.

For example, there are known dental based non-invasive fiducial array such as those described in U.S. Pat. Nos. 6,096,048 and 6,223,067 and United States Patent Application Publication No. US2004/0015176. However, often, it is desirable that a dental based fiducial array can be removed from a patient's mouth (or an animal's mouth) and re-installed later very accurately to the original position in the mouth. The accuracy is crucial to the precision of image scan and for registration of the imaged anatomy to the scanned images. Further, structural members of a fiducial array may not always be suitably positioned for patients of all ages. Similarly, different geometries of animals of different species may also cause interferences with part of the geometries or require re-positioning of fiducial markers to different locations to closely follow the location of brain regions.

The forgoing creates challenges and constraints for a fiducial array for providing easily locatable fiducial points for neuronavigation and other similar procedures. There is therefore a need for a fiducial array as compared to the existing art. It is an object of the present invention to mitigate or obviate at least one of the above mentioned disadvantages.

### SUMMARY OF INVENTION

The invention relates generally to apparatus and devices for neuronavigation. More particularly, a non-invasive dental based fiducial array is disclosed. One aspect of the present invention involves a fiducial array that includes a base support member, one or more removable and repositionable hubs supported by the base support member and a stopper spaced from and supported by the base support member that engages a part of the head of a patient or an animal and cooperates with the base support member to immobilize the fiducial array relative to maxillary teeth. An anchoring site is formed on the base support member to accept a custom made dental impression to precisely position the base support member relative to the maxillary teeth. The hubs have removable marker base attached thereto for supporting fiducial markers.

In an aspect of the invention, a non-invasive, dental-based fiducial array is provided. The fiducial array includes a base support member, a support arm, and one or more marker bases removably and re-positionably mounted to the support arm, each of said one or more marker bases being adapted for mounting a fiducial marker. The base support member is shaped to be received in oral cavity of a person or an animal and includes an anchoring site for accepting a customized dental impression. The support arm is pivotally secured to the base support member.

One feature of the aspect of the invention provides a fiducial array that further includes a stopper spaced from and re-positionably mounted to the base support member. The stopper is sized to engage the head of the person or the animal and cooperating with the base support member to immobilize the base support member relative to the head. As a further feature, the fiducial array also includes a mounting post slidably coupled to the base support member and a mounting block releasably secured to the mounting post, the stopper being re-positionably secured to the mounting block.

According to another feature of the aspect of the invention, the fiducial array further includes a removable hub mounted on the support arm, the one or more marker bases being mounted to the support arm through the removable hub.

According to yet another feature of the aspect of the invention, the fiducial array further includes a connection member that pivotally connects the support arm to the base support member. According to, a further feature, the connection member includes two pivot connections and the two pivot connections have non-parallel pivot axes.

In other aspects the invention provides various combinations and subsets of the aspects described above.

### BRIEF DESCRIPTION OF DRAWINGS

For the purposes of description, but not of limitation, the foregoing and other aspects of the invention are explained in greater detail with reference to the accompanying drawings, in which:

FIG. 1 is a perspective view showing a dental based fiducial array;

FIG. 2 is a cross-sectional view showing a connection link that pivotally connects support arm of the fiducial array of FIG. 1 to its base support member;

FIG. 3 illustrates in a perspective view a base support member of the fiducial array shown in FIG. 1;

FIG. 4 shows in a perspective view an upright post;

FIG. 5 shows in a perspective view a support arm;

FIG. 6 shows in a perspective view an alternative support arm;

FIG. 7 shows in a perspective view a removable hub (with some spokes and fiducial marker discs removed for better illustration) pivotally supported on a base plate of the fiducial array shown in FIG. 1;

FIG. 8 is a perspective view showing a dental based fiducial array that includes a stopper;

FIG. 9 is a perspective view a stopper of the fiducial array of FIG. 8;

FIG. 10 shows in a cross-sectional view a stopper that includes a slide mount secured to a mounting post which is in turn secured to a base plate;

FIG. 11 shows in a perspective view another construction of a dental-based fiducial array; and

FIG. 12 shows in an exploded view the dental-based fiducial array of FIG. 11.

### DETAILED DESCRIPTION OF EMBODIMENTS

The description which follows and the embodiments described therein are provided by way of illustration of an example, or examples, of particular embodiments of the principles of the present invention. These examples are provided for the purposes of explanation, and not limitation, of those principles and of the invention. In the description which follows, like parts are marked throughout the specification and the drawings with the same respective reference numerals.

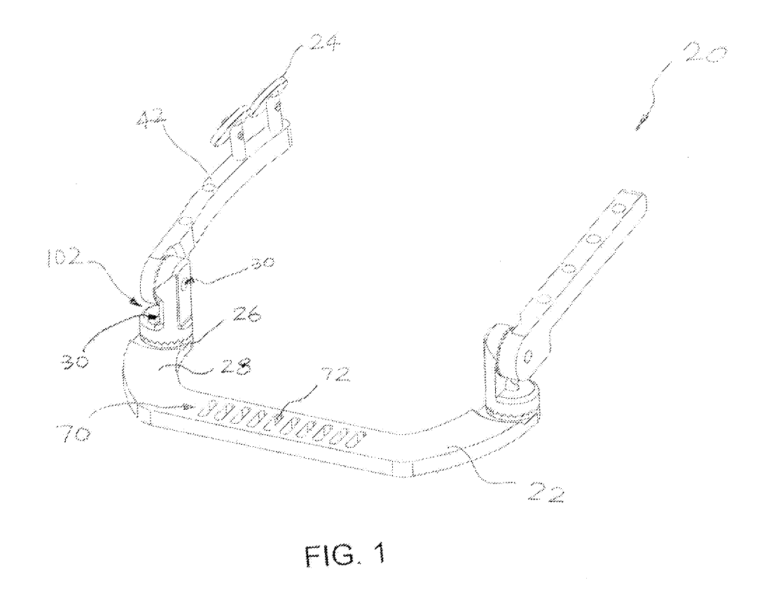

Referring to FIG. 1, there is shown an embodiment of a dental based fiducial array, generally indicated by reference number 20. Fiducial array 20 has a base support member such as a base plate 22. Base plate 22 may have a general U-shape, or some other shapes to be received in oral cavity of a person or an animal so that the person or animal may bite part of the base plate to immobilize the base plate with respect to a location of head, such as upper or lower jaw, or maxillary teeth or upper palate, while some other part of the base plate can be exposed for supporting other components of the fiducial array, without interfering with the anatomy of the oral cavity or the head. Base plate can be sized to fit human and/or animal mouth sizes. It will be appreciated, however, that the base plate may also take other shapes, such as an arc, a plate having an inner curved edge to accommodate the jaw anatomy, among others. As will be described in detail later, base plate provides an anchor point for precise positioning of the fiducial array relative to a location of the head, such as maxillary teeth of a person or animal. The exposed portion of the base plate can also be used for positioning the person or the animal's head relative to other medical instrument where needed.

The base plate 22 has an anchoring site 70 for receiving a custom made dental impression. The anchoring site 70 has a plurality of slots 72 (or holes or other anchoring arrangements) formed in the base plate 22. A dental impression of maxillary teeth (and if desired, upper palate) of a patient or an animal is formed from a thermo-moldable dental plastic and includes a plurality of projections to be received in the plurality of slots 72. Conveniently, the thermo-moldable dental plastic may be placed on the base plate 22 with the maxillary teeth pressed against the base plate to make the dental impression. The pressure applied tends to create a plurality of projections that extend into and match the shape and location of the plurality of slots, thus providing desirable fitting between the dental impression and the base plate.

One or more removable marker support, such as marker discs 24 are removably and pivotally joined to and supported by the base plate 22. As will be described in detail later, each marker disc 24 is adapted for mounting a marker, which provides locatable fiducial points for neuronavigation. A support arm 42 may be provided for mounting the marker discs. A connection member, such as connection link 102 that includes a pivot connection or connections 30, pivotally joins the support arm 42 to the base plate. Pivot connections allow the marker discs to be repositioned relative to the base plate, when required. FIG. 1 shows a U-shaped base plate. Connection link is mounted to the base plate 22 at the distal ends 26 of the legs 28 of the U. This allows the markers to be placed as close to the brain region as possible, often of interest to a neurosurgeon.

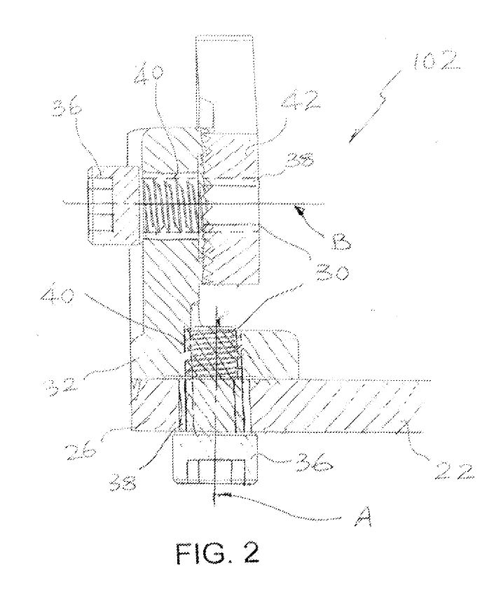

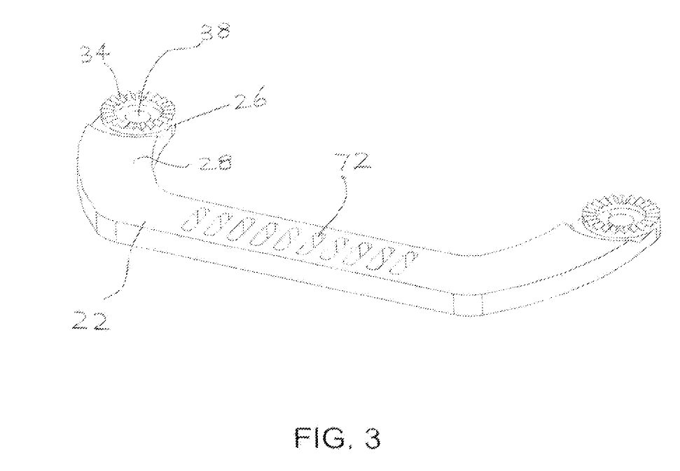

Connection link 102 shown in FIG. 1 and FIG. 2 has two pivot connections 30, each pivot connection having a pivot axis A, B, and pivot axes A, B being non-parallel. Referring to FIGS. 1 to 5, an upright post 32 is pivotally joined to the base plate adjacent its distal end 26. The upright post 32 has a toothed connection surface 34 to mate with a corresponding toothed connection surface 34 formed on the base plate 22 (FIG. 3). A securing screw 36 or such other appropriate fasteners can be used to pass through a throughhole 38 formed in the base plate and threaded into a threaded hole 40 formed in the upright post to releasably secure the upright post to the base plate 22. As can be more clearly seen in FIG. 2, upright post 32 is pivotable about rotational axis A of the securing screw 36. Thus, connection between the upright post to base plate provided by the securing screw 36 allows the upright post 32 to pivot about the pivot axis A and provides the first pivot connection 30. Tightening the securing screw 36 urges the toothed connection surfaces 34 toward each other until stopped and thereby fixes the orientation of the upright post 32 about its pivot axis. The toothed connection surfaces 34 define a series of pre-selected angular positions and facilitate precise angular positioning of the upright post.

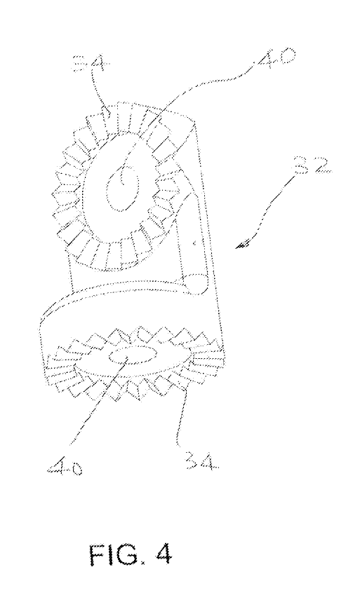

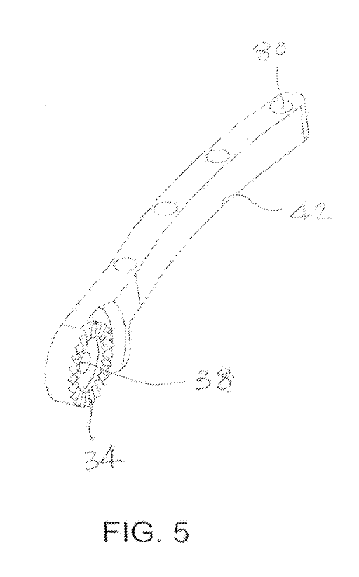

A support arm 42 is pivotally joined to the upright post 32 at a location spaced from the base plate. A securing screw 36 is used to provide a second pivot axis B and to secure the support arm 42 to the upright post 32. Similarly, matching toothed connection surfaces 34 may be formed on support arm 42 and upright post 32, as shown in FIGS. 4 and 5, for precise angular positioning of support arm 42 relative to upright post 32. Throughhole 38 formed in support arm 42 and threaded hole 40 in upright post 32 allow a securing screw 36 to pass through and secure the support arm to the upright post. The connection between support arm 42 and upright post 32 provides the second pivot connection 30, i.e., a pivot connection pivotable about pivot axis B. Of course, a threaded hole may also be formed in support arm 42 and a throughhole formed in upright post 32, or throughholes be formed in both support arm 42 and upright post 32, in which case a nut and a bolt will be required.

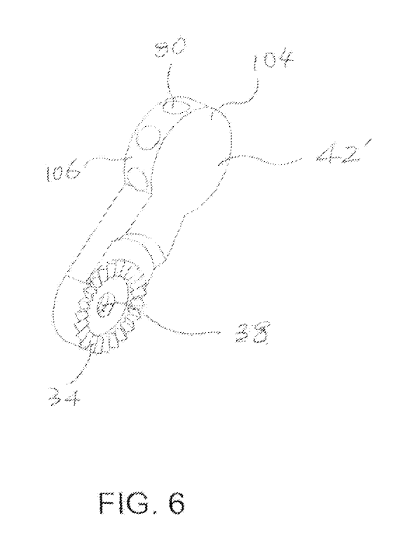

Support arm 42 may be adapted for direct attachment of fiducial marker discs 86. FIG. 5 illustrates a support arm 42 that has a series of spoke holes 80 formed along one of its sides, each of which is suitably sized for accepting a spoke 84 that supports a fiducial disc 86 thereon. Support arm may be straight or suitably curved as seen in FIG. 1. One support arm 42 shown in FIG. 1 is suitably curved so that the markers can be positioned as close to the head of a patient or animal as possible. FIG. 6 illustrates another example of a support arm for direct attachment of fiducial marker discs. Support arm 42′ shown in FIG. 6 has a round terminal end 104. A series of spoke holes 80 are formed on the periphery side 106 of the terminal end 104 for accepting spokes 84 to mount fiducial marker discs 86 thereon.

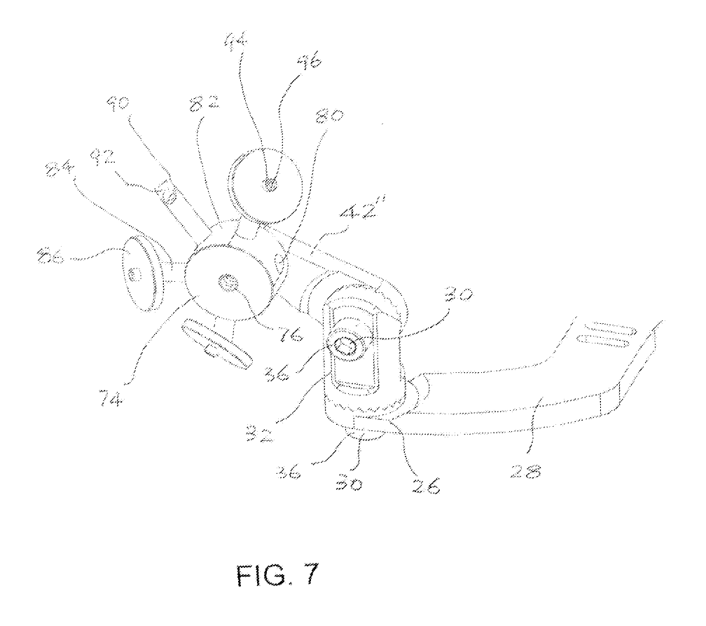

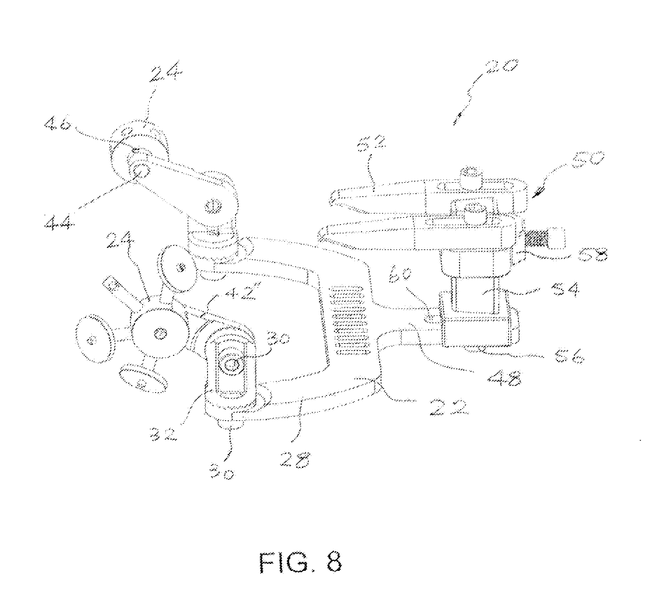

As yet another alternative, instead of forming a round terminal end at the distal end of support arm, one or more removable hubs 24 may be provided. As the pivot axes A, B of the two pivot connections 30 are in different planes and oriented differently, removable hub 24 attached to support arm also has at least two degrees of freedom. Referring to FIGS. 7 and 8, a removable hub 24 can be attached to support arm 42″ by way of a short arm 44. A screw 46 or pin secures the removable hub 24 to the short arm 44. Each removable hub 24 has a generally cylindrical body 74, with a central hole 76 for receiving a short arm 44 of support arm 42″ and a plurality of spoke holes 80 formed on and angularly spaced along the side surface 82 of the cylinder. A spoke 84 is received in a spoke hole 80 and supports a fiducial marker base 86. Spoke holes 80 may be formed along a generally radial direction and transverse to the longitudinal axis of the cylinder. They may also be at an angle different from 90° with the longitudinal axis of the cylinder. For example, the angle may be between 60° and 90°.

The orientation of the removable hub 24 relative to the support arm 42″ may be adjusted by turning it around short arm 44 prior to tightening the securing screw 36. Alternatively, short arm 44 may have a non-circular cross-section, with the hub having a hole of matching cross-sectional shape for receiving the short arm. For example, the cross section may be a hexagon, or some other polygon. The non-cylindrical cross-section allows accurate orientation of the hub relative to the support arm and any subsequent repositioning. With a cross-sectional shape such as hexagon that has rotational symmetry, stepped adjustment of relative orientation of the hub will also be possible. Of course, other means may be provided for accurate repositioning, such as markings on the hub and the short arm or the support arm.

As noted, a fiducial marker base 86 is supported at the top end 90 of a spoke 84. Fiducial marker base 86 and spoke 84 may be individually formed and then joined together, or integrally formed as one piece. Fiducial marker base shown in FIG. 1 and FIG. 7 has the shape of a disc, though other shapes are also possible. The fiducial marker disc 86 is secured to the spoke. The top end of the spoke has a supporting surface 92. The orientation of supporting surface 92, i.e., the normal of the surface, may not be aligned with the longitudinal axis of the spoke 84. Instead, the supporting surface may be inclined, with the normal and the longitudinal axis of the spoke 84 forming a non-zero inclination angle. With markers positioned on supporting surfaces at such an inclination angle, the markers will not be co-planar, which may be desirable for certain applications. The inclination angle may be conveniently selected to be 30°, though it may be an angle up to 45°, or zero (i.e., not inclined).

A fiducial marker disc 86 is adapted for accepting fiducial markers (not shown). At the center of the fiducial marker disc 86 there may be provided a small nipple 94, for fitting a self-adhesive multi-modality marker. A receiving hole may be formed in spoke 84 as shown in FIG. 6 for receiving the small nipple. The nipple may have a nipple hole 96 (about 1.5 mm), into which the distal tip of a neuronavigation pointer (not shown) can be touched to co-register the location of the supported fiducial marker to scan imaging data.

Conveniently, a stopper 50 is provided for immobilizing the base plate relative to a location of the head, such as upper or lower jaw. Referring to FIG. 8, a base plate may be configured for supporting the stopper 50, for example, by integrally forming an extended leg 48 at the bottom segment of U for mounting the stopper thereon. A stopper may be in the form of an eye bar 52 or a set of eye bars 52. Stopper 50 engages a region of the head of the patient or the animal. Eye bar 52 is spaced from base plate. Its height relative to the base plate 22 is adjustable so that appropriate pressure can be applied by the cooperating stopper and base plate 22 to increase the position retaining power of the fiducial array. As will be appreciated, with an extended leg 48 integrally formed with base plate 22, the extended leg 48 may also be used for supporting other instruments on the base plate, or for attaching the base plate to a support structure or surface to immobilize head during imaging or surgery procedure, where desirable or required.

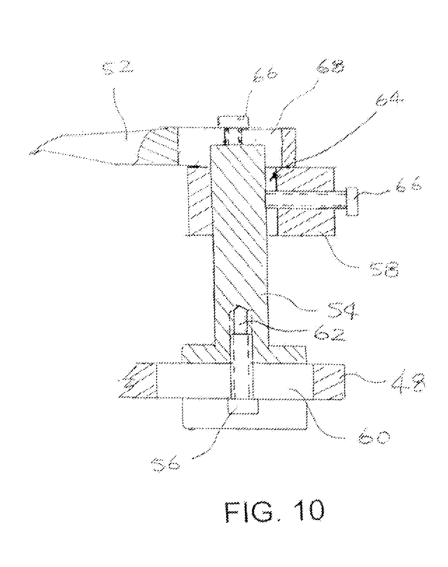

Referring to FIGS. 8, 9 and 10, a mounting post 54 is secured to the extended leg 48 by a securing bolt 56. Eye bar 52 (or set of eye bars) is supported by a slide mount 58 that is slidably attached to the mounting post for adjusting height of the eye bar 52 relative to the base plate 22. A slot 60 is formed in the extended leg 48. A bolt hole 62 is formed in the mounting post 54. A securing bolt 56 passes through the slot 60 and is threaded into the bolt hole 62 of the mounting post to secure the mounting post to the base plate. The slot 60 allows the mounting post 54 to be positioned at any location along the slot as may be desired. Thus, both height of eye bar 52 and distance of eye bar 52 along extended leg 48 can be adjusted and eye bar 52 can be re-positioned by adjusting its height and distance as desired or needed.

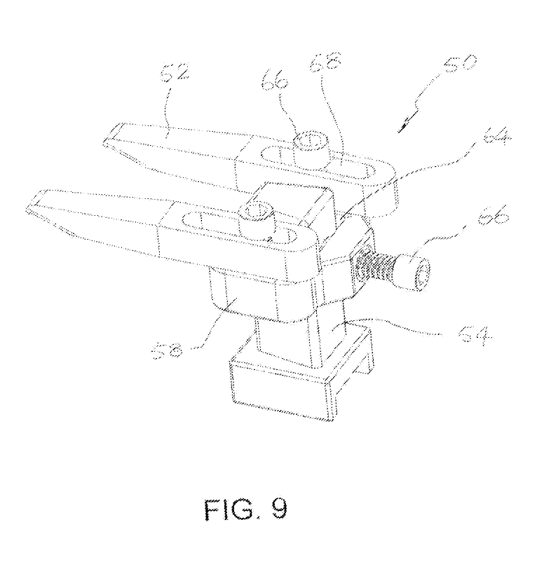

The transverse cross section of mounting post 54 may have any suitable shape. FIGS. 8 and 9 show an example that has a trapezoidal shape. The slide mount 58 has a corresponding trapezoidal through hole 64 for the trapezoidal shaped mounting post 54 to pass therethrough. A tightening screw 66, or other suitable engagement means, is provided for pulling the mounting post towards the slide mount, along a direction from the longer side to the shorter side of the trapezoidal. Eye bar 52 has an eye bar slot 68 for a tightening screw 66 to pass through and to secure the eye bar to the slide mount 58. The eye bar slot allows the eye bar to be positioned differently as the eye bar slides relative to the slide mount 58.

Of course, although a stopper in the form of a height-adjustable eye bar is described, the stopper may take any other suitable form. For example, a strap system (or even much simpler surgical tape) can be used to replace the eye bar for immobilizing the base plate relative to the maxillary teeth (or upper palette).

The device described herein allows it to be applied to patients of different ages and also to animals of different species. To use the device for a patient (or an animal), a suitably sized base plate 22 is selected and placed in the mouth of the patient or animal. A suitable mass of thermo-moldable dental plastic, already appropriately heated, can be placed on the base plate for making a customized dental impression. Once solidified, the dental impression tends to be securely attached to the base plate, which will provide accurate anchor points when the fiducial array is reinstalled in the mouth of the patient or animal, from whom the dental impression is made.

One or more marker discs are installed for mounting fiducial markers. Position of the marker discs, or fiducial markers, can be adjusted by rotating (i.e., pivoting) the upright post 32 and the support arm 42. The pivot connections 30 provided by the upright post and the support arm allow the marker discs, or removable hubs if used for supporting marker discs, to be placed as close to patient's head (or the animal's head) as possible and close to the brain area which is of interest to a neurosurgeon. Once the marker discs or the removal hubs are suitably positioned, the securing screws 36 can be tightened. It is possible that one or more of the spokes 84 or fiducial marker discs 86 may interfere with some parts of the patient's (or the animal's) anatomy. The interfering fiducial marker disc or discs can be removed or the interfering spoke(s) be removed to avoid interference. The position of the removable hub or hubs and the fiducial marker discs may be recorded or left unchanged for future use.

When the fiducial array needs to be reinstalled, the custom made dental impression is matched with the dental structures of the patient or animal, such as formation of maxillary teeth or upper palate, to ensure accurate positioning of the base plate 22 and the fiducial marker discs 86 carried by the base plate. The positions of fiducial marker discs may be further fine tuned should there be any relocation of these marker discs since last image scan. The height and position of stoppers 50 can be suitably adjusted to immobilize the fiducial array 20 during the surgical, imaging or other medical procedures.

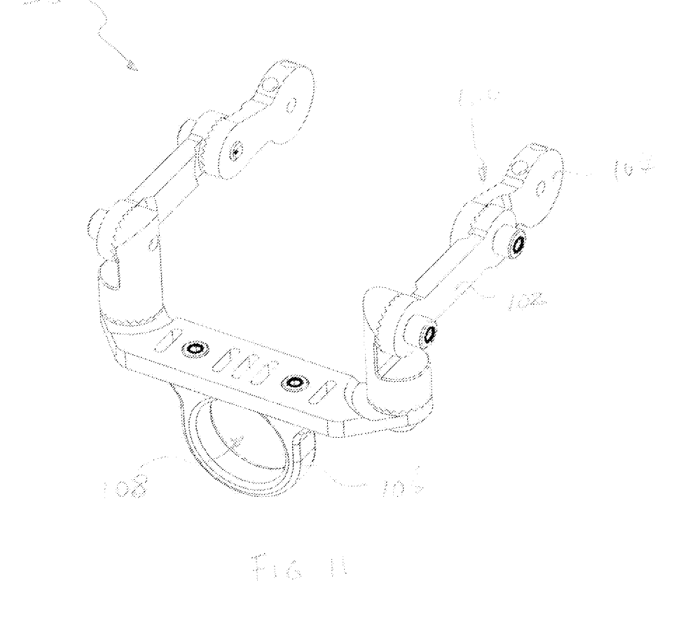

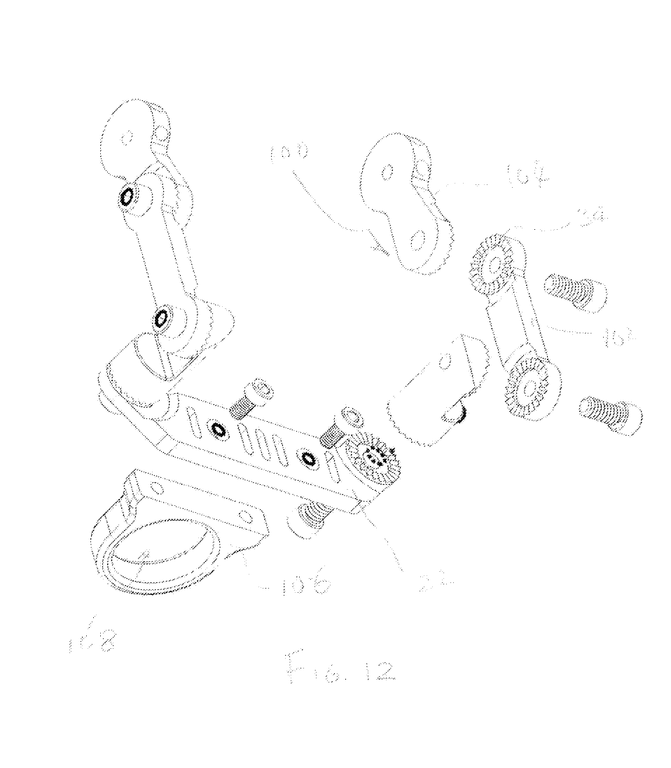

FIGS. 11 and 12 show in a perspective view and an exploded view another construction of a dental-based fiducial array 20″. Fiducial array 20″ is similar in construction to fiducial array 20 shown in FIG. 1. Unlike the single-piece construction of support arm 42 of fiducial array 20, support arm 100 has a multi-section construction. As shown in FIGS. 11 and 12, support arm 100 has a first section 102 and a second section 108, pivotally joined to each other. Toothed connection surfaces 34 can be provided at each pivot connections to define a series of pre-selected angular positions and facilitate angular positioning of neighboring sections. Although only two sections are shown in FIGS. 11 and 12, it will be understood that more sections may be used where appropriate and feasible. The distal section, or the second section 104 in this case, is shown to be the same as the short support arm 42 that has a round terminal end for mounting fiducial marker discs as described in connection with fiducial array 20 and illustrated in FIG. 6. Support arm 100 may also include holes 80, similar to that shown in FIG. 1, or hubs 24 similar to that shown in FIG. 7, for mounting fiducial marker discs.

Fiducial array 20″ also includes an attachment 102 which has a throughhole 104 defined therein. The attachment is shown to be secured to base plate 22 by threaded nuts, though it may also be secured using any other suitable fasteners. The attachment may also be integrally formed with base plate 22. When the fiducial array is placed in the mouth of a patient or animal, the attachment provides support so that the mouth may remain open for longer duration without causing excessive fatigue. The throughhole 108 can be used to accommodate endotrached tube (“ET tube”) or other tubing during surgery. Tubings that pass through the throughhole 108 thus are protected and the mouth of the patient or the animal would not be closed so as to squeeze the ET tube or other catheters or probes in the mouth. The attachment 102 can also be used together with a strap to keep the fiducial array 20″ in position in the mouth of an animal. For example, the fiducial array 20″ may be placed in an animal's mouth so that the lower jaw and the maxillary teeth can close on the attachment 104 and the dental impression secured to base plate 22. A strap may be provided to help keep the mouth closed.

Various embodiments of the invention have now been described in detail. Those skilled in the art will appreciate that numerous modifications, adaptations and variations may be made to the embodiments without departing from the scope of the invention. Since changes in and or additions to the above-described best mode may be made without departing from the scope of the invention, the invention is not to be limited to those details but only by the appended claims.

What is claimed is:

1. A dental-based fiducial array for providing re-positionable fiducial points, required by neuronavigation, the fiducial array comprising:
a base support member, the base support member being shaped to be received in oral cavity of a person or an animal and including an anchoring site for accepting a customized dental impression,
a support arm, said support arm being pivotally joined to the base support member, and one or more marker bases removably and re-positionably mounted to the support arm, each of said one or more marker bases being adapted for mounting a fiducial marker.

2. The fiducial array of claim 1, further comprising a stopper spaced from and re-positionably mounted to the base support member, said stopper being sized to engage the head of the person or the animal and cooperating with the base support member to immobilize the base support member relative to the head.

3. The fiducial array of claim 2, further comprising a mounting post slidably coupled to the base support member and a mounting block releasably secured to the mounting post, the stopper being re-positionably secured to the mounting block.

4. The fiducial array of claim 1, wherein said base support member has a throughhole defined therein for tubings to pass therethrough.

5. The fiducial array of claim 1, wherein said base support member includes an attachment having said throughhole defined therein.

6. The fiducial array of claim 1, further comprising a removable hub attached to the support arm for mounting the one or more marker bases, the one or more marker bases being mounted to the support arm through the removable hub.

7. The fiducial array of claim 6, wherein said removable hub has a plurality of holes formed thereon for accepting the one or more marker bases, each of said one or more marker bases having a post to be received in one of said plurality of holes.

8. The fiducial array of claim 1, wherein said support arm has a plurality of holes formed thereon for accepting the one or more marker bases, each of said one or more marker bases having a post to be received in one of said plurality of holes.

9. The fiducial array of claim 8, wherein said post defines a longitudinal axis, said each marker base having a support surface for mounting said fiducial marker, normal direction of said support surface and said longitudinal axis forming a non-zero inclination angle.

10. The fiducial array of claim 9, wherein the non-zero inclination angle is less than 45°.

11. The fiducial array of claim 1, wherein said support arm comprises a plurality of sections pivotally joined to each other.

12. The fiducial array of claim 11, wherein said pivotally joined sections are configured to be releasably secured to one of a plurality of pre-selected angular positions.

13. The fiducial array of claim 12, wherein each of said pivot connections has matching toothed connection surface formed on, said toothed connection surfaces defining said plurality of pre-selected angular positions.

14. The fiducial array of claim 1, further comprising a connection member, said connection member pivotally joining said support arm to the base support member.

15. The fiducial array of claim 14, wherein said connection member includes a first pivot connection and a second pivot connection, said first and second pivot connections having non-parallel pivot axes, said first connection connecting said connection member to the base support member, said second connection connecting said connection member to said support arm.

16. The fiducial array of claim 15, said first and second pivot connections each having a plurality of pre-selected angular positions and each of said pivot connections being configured to be releasably secured to one of said plurality of angular positions.

17. The fiducial array of claim 16, wherein each of said first and second pivot connections has toothed connection surface formed on said connection member, said toothed connection surface defining said plurality of pre-selected angular positions.

18. The fiducial array of claim 1, wherein said anchoring site includes a plurality of slots for accepting and securing thereto said dental impression.
